(19) United States
(12) Patent Application Publication
(10) Pub. No.: US 2007/0276318 A1
(43) Pub. Date: Nov. 29, 2007

(54) Iontosonic-microneedle applicator apparatus and methods

(75) Inventor: Julian L. Henley, New Haven, CT (US)

Correspondence Address: STERNE, KESSLER, GOLDSTEIN & FOX P.L.L.C., 1100 NEW YORK AVENUE, N.W., WASHINGTON, DC, 20005, US

(73) Assignee: MIT, LLP, Miami, FL (US)

(21) Appl. No.: 11/599,386

(22) Filed: Nov. 15, 2006

Related U.S. Application Data
(60) Provisional application No. 60/808,546, filed on May 26, 2006.

(51) Int. Cl. A61N 1/30; A61B 17/20

(52) U.S. Cl. 604/20; 604/22

(57) ABSTRACT

Novel multichannel ionosonic devices with microneedle arrays incorporated into the devices for transdermal and intradermal delivery are described. In an embodiment, the ionosonic device includes a multichannel ionophoretic driver used in combination with a multichannel dispersion electrode integrated with ultrasonic elements and microneedle array elements mounted on a single application electrode. The ionosonic-microneedle transdermal device can be configured in a variety of shapes and structural flexibility for the treatment of skin and systemic disorders through the intradermal and transdermal delivery of one or more of a variety of therapeutic and modulating agents. Because of enhanced transdermal penetration this device offers the transdermal delivery of therapeutic peptides is getting closer to reality. The devices described herein deliver the therapeutic agents to the targeted diseased area as well as systemic levels obviating the need for oral ingestion, the associated side effects and as in the case of peptides bypasses the hydrolyzing digestive enzymes that make such agents ineffective when taken by mouth.

### BACKGROUND OF THE INVENTION

1. Field of the Invention

The present invention relates to applicator devices and methods.

2. Background Art

It has long been desirable to apply medication or other agents through skin, nail, or other biological tissue. This allows medication or other agents to be delivered more directly to affected topical surface areas and to targeted regions within a person's body. Delivering a drug across the skin or other tissue membrane, including transdermal or intradermal drug delivery, can also be advantageous in many applications where it is desirable to avoid problems associated with oral ingestion and drug delivery through the acidic environment of a stomach, or discomfort and hygienic issues involved in long needle delivery applications.

Various methods have evolved to apply medication or other agents across skin. Iontophoresis has existed for several centuries as a way to apply medication locally through a patient's skin and to deliver medicaments to the eyes and ears. The application of an electric field to the skin is known to greatly enhance the skin's permeability to various ionic agents. The use of iontophoretic techniques has obviated the need for hypodermic injection of certain medicaments, thereby eliminating the concomitant problems of trauma, pain and risk of infection to the patient.

Iontophoresis involves the application of an electromotive force to drive or repel oppositely charged ions through the dermal layers into the area to be treated; either into the surrounding tissues for localized treatment or into the circulatory system for systemic treatment. Positively charged ions are driven into the skin at the anode while negatively charged ions are driven into the skin at the cathode. One readily observed benefit of transdermal iontophoretic drug delivery is the increased efficacy of the drugs delivered in this fashion. Studies have shown increased skin penetration of drugs at anodic or cathodic electrodes regardless of the predominant molecular ionic charge. This effect is mediated by polarization and osmotic effects. Regardless of the electrical charge on the medicament employed, two electrodes are used in conjunction with the patient's skin to form a closed circuit to promote the penetration or absorption of the medicament through the skin underlying the working electrode.

The present inventor has disclosed and patented iontophoretic devices that have been shown to effectively treat herpetic infection of the mucocutanous junction. A number of studies demonstrate that a single channel iontophoretic device and electrode design according to previously specified parameters has been efficacious in treating, and rapidly attenuating, a herpetic skin infection in humans with a single 3-10 minute application of an antiviral agent. Studies have also demonstrated that such a device is able to drive a significantly greater amount of Acyclovir into the dermis as compared to topical application of the antiviral agent.

Such single channel therapeutic devices apply an ionophoretic current and carry the therapeutic agent (in this particular case, Acyclovir), into the lesion where the herpes virus is replicating. This approach has proven itself, after numerous studies, to be greatly efficacious in effectively treating the viral herpetic infection at the mucocutaneous junction.

Different approaches have been used to further improve the performance of iontophoretic devices. One approach is to use a multichannel dispersive electrode. For example, U.S. Pat. No. 5,160,316, issued to the instant inventor and incorporated in its entirety herein by reference, describes the use of a multichannel dispersive electrode. Each channel is driven by separate electronic circuits to assure wide dispersion and enhanced penetration of medicament. Such wide field electrodes not only can cover a wide area of body without succumbing to “tunneling effects” but provide sufficient skin penetration to function as a systemic drug delivery system. A second approach is to add ultrasonic elements to iontophoretic devices (this combination being referred to herein as ionosonic devices). For example, U.S. Pat. No. 5,658,247, issued to the instant inventor, describes a multichannel ionophoretic driver mounted on same application electrode with ultrasonic elements for enhanced intradermal delivery of therapeutic agents. Such ionophoretic devices with ultrasonic elements have not recognized nor used microneedle assemblies.

Drug delivery devices have also used microneedle assemblies to improve transport across tissue barriers. See, U.S. Pat. No. 6,334,856 issued to Allen et al. Drug delivery devices have also been enhanced with iontophoresis and microneedle assemblies. See, U.S. Pat. No. 6,3312,612 issued to Sherman et al. Microneedle arrays can improve transdermal delivery by penetrating the stratum corneum of the skin with the sharp elements. Since sensory pain receptors are located within the dermal layer of the skin using multiple rigid sharp tipped projections in the range of 10 to 350 microns has been found by numerous investigators to make the dermis (skin) more permeable and therefore helpful in technologies aimed at transdermal delivery or reverse transdermal sampling for diagnostic and measurement purposes. Such iontophoretic-microneedle assemblies, however, have neither been recognized nor used ultrasound or multichannel electrodes.

What is needed are improved iontophoretic-ultrasonic devices and methods.

### BRIEF SUMMARY OF THE INVENTION

The present invention provides novel delivery devices and methods utilizing iontonsonic and microneedle technologies. These delivery devices and methods can be used in a wide variety of embodiments and applications including, but not limited to, intradermal or transdermal delivery of one or more therapeutic agents toward or into a tissue (such as a skin or nail).

In an embodiment, an applicator can ionosonically drive an agent toward a targeted portion of a mammal's body. The applicator includes one or more piezoelectric elements, a plurality of iontophoresis electrodes, and a microneedle assembly. The microneedle assembly has a plurality of microneedles that can be placed at or near the targeted portion of a mammal's body and in direct contact with skin or tissue. The one or more piezoelectric elements are coupled to the microneedle assembly. The plurality of microneedles move and vibrate the tissue in contact with the applicator in response to the changes in the piezoelectric element(s). Electrical current can be applied to the plurality of iontophoresis electrodes to further drive the agent toward the targeted portion of the mammal's body. Such electromechanical coupling between the piezoelectric, ionophoretic and microneedles jointly effect the penetration to a greater degree than any of the components separately. Such combined coupling of the aforementioned technologies working together and concurrently on the applicator electrode lead to delivery flux benefits that are greater and more efficacious than any of the technologies working alone or separately.

According to a feature, the microneedle assembly further includes a plurality of microchannels. The plurality of microchannels can be interspersed between the plurality of microneedles.

According to another embodiment, the iontophoresis electrodes are arranged at or near respective groups of microneedles and microchannels. In a multichannel embodiment, each iontophoresis electrode can be separate channel electrically isolated from other channels. A plurality of current drivers can be coupled to respective ones of the plurality of iontophoresis electrodes. In this way, each iontophoresis electrode comprises a separate electrical channel driven by a corresponding current driver.

According to a further embodiment, an applicator includes a support member arranged to support one or more piezoelectric elements relative to the microneedle assembly as well as the plurality of iontophoresis electrodes. In one example, a plurality of iontophoresis electrodes are imbedded within the support member.

In some embodiments, an agent can be included in a medicament. The medicament can be included in the applicator as part of a separate medicament layer, as part of the support member, as part of the microneedle assembly (for example in perforations in the microneedle assembly), or applied externally to exterior of the microneedle assembly. In one embodiment the medicament is saturated into the microchannels dispersed among the projecting microneedles. In each case, the agent is released or moved when the plurality of microneedles move and vibrate tissue in contact with the applicator in response to the at least one piezoelectric element, and when electrical current is applied to the plurality of iontophoresis electrodes to further drive the agent toward the targeted portion of the mammal's body.

In an embodiment, an ionosonic intradermal drug delivery device adapted to releasably attach to a mammal's body so that a skin-contacting surface of the device is adjacent to a targeted portion of the mammal's skin or nail. The device is operable for ionosonically driving a medicament across a skin-contacting surface of the device into a targeted portion of the mammal's skin or nail. The device includes (a) a medicament carrying layer in fluid communication with the skin-contacting surface of the device comprising a porous sheet impregnated with a medicament containing fluid, (b) an iontophoresis electrode in electrical communication with the medicament carrying layer providing means for iontophoretically driving the medicament into the targeted portion of the mammal's skin or nail(s) wherein the iontophoresis electrode comprises a plurality of electrode channels, each electrode channel of the plurality of electrode channels being electrically isolated from other electrode channels, the plurality of electrode channels having electrical connection means connected thereto adapted for simultaneous electrical connection of selected electrode channels to the same or different current sources, the each electrode channel being in electrical communication with the medicament carrying layer; (c) at least one piezoelectric element affixed to the iontophoresis electrode and overlying at least one of the iontophoresis electrode channels; and (d) at least one perforated non or partially conductive microneedle array layer interposed between the skin and iontophoretic dispersion electrode.

In an embodiment, a method for the transdermal or intradermal self-administration of a medicament into a targeted portion of the skin or nail of an individual is provided. The method includes (a) presenting an ionosonic intradermal drug delivery device; (b) impregnating a porous sheet with a medicament and placing the porous sheet in contact with the targeted portion of the individual's skin or nails; and (c) causing an electrical current to flow through the electrode channels of the iontophoresis electrode to drive the medicament into the targeted portion of the skin or nail.

Further embodiments, features, and advantages of the present invention, as well as the structure and operation of the various embodiments of the present invention, are described in detail below with reference to the accompanying drawings.

### BRIEF DESCRIPTION OF THE FIGURES

The accompanying drawings, which are incorporated herein and form a part of the specification, illustrate the present invention and, together with the description, further serve to explain the principles of the invention and to enable a mammal skilled in the pertinent art to make and use the invention.

FIGS. 1A and 1B show a multichannel iontophoretic applicator having a plurality of ultrasonic elements and a microneedle assembly that can be used to treat large dermal areas according to an embodiment of the present invention. FIG. 1A is a top plan view of a multichannel iontophoretic applicator having a plurality of ultrasonic elements that can be used to treat large dermal areas. FIG. 1B is a cross-sectional view of the applicator of FIG. 1A showing a microneedle assembly with microchannels according to an embodiment of the present invention.

FIG. 2 is a top, schematic, plan view of a conventional multichannel iontophoretic-ultrasonic applicator arranged in a band configuration for intradermal drug delivery that can be further adapted to include the microneedle assembly of FIG. 1B according to a further embodiment of the present invention.

FIG. 3 is a block circuit diagram of a conventional iontophoretic-ultrasonic (ionosonic) medicament applicator's electrical control circuit that can be used with the applicators of FIGS. 1 and 2 either as a separate power and control unit or integrated into a single unit.

FIG. 4A is a palmar view of a glove applicator having a plurality of iontophoresis electrodes and return (neutral) electrodes disposed thereon that can be further adapted to include the microneedle assembly of FIG. 1B according to a further embodiment of the present invention. FIG. 4B is a perspective view of a mammal employing the applicator device of FIG. 4A to self-administer a cosmetic or therapeutic agent to the skin of the face thereafter the agent to be delivered to intradermal tissue.

FIG. 5 is a schematic view of a bootie applicator having a plurality of iontophoresis electrodes and ultrasonic elements disposed thereon for delivering an antifungal agent between the toes or into toenails that can be further adapted to include the microneedle assembly of FIG. 1B according to a further embodiment of the present invention.

FIG. 6 is a schematic view of a mask applicator having a plurality of iontophoresis electrodes and ultrasonic elements disposed thereon for the intradermal delivery of a cosmetic or therapeutic agent to the skin of the face.

The present invention will be described with reference to the accompanying drawings. The drawing in which an element first appears is typically indicated by the leftmost digit(s) in the corresponding reference number.

### DETAILED DESCRIPTION OF THE INVENTION

The present invention relates to applicator devices and methods that can apply one or more agents of any type including but not limited to therapeutic agent(s). While specific embodiments, configurations and arrangements are discussed, it should be understood that this is done for illustrative purposes only. A person skilled in the pertinent art will recognize that other embodiments, configurations and arrangements can be used without departing from the spirit and scope of the present invention. It will be apparent to a mammal skilled in the pertinent art that this invention can also be employed in a variety of other applications.

Medicaments for use in the present invention can be any type of medicament including, but not limited to: any medicament or combination of medicaments for treating or preventing microbial infection of skin or nail, treating or preventing acne, treating psoriasis, treating or preventing eczema or contact dermatitis or atopic dermatitis, treating or preventing onychomycosis, treating actinic keratoses, treating skin cancer, treating a wound, or treating an infection by a human papilloma virus. The medicament can be any type of medicament including, but not limited to, an anesthetic, therapeutic drug, or a non-therapeutic agent. The medicament can be in any form including, but not limited to, any type of cream or liquid.

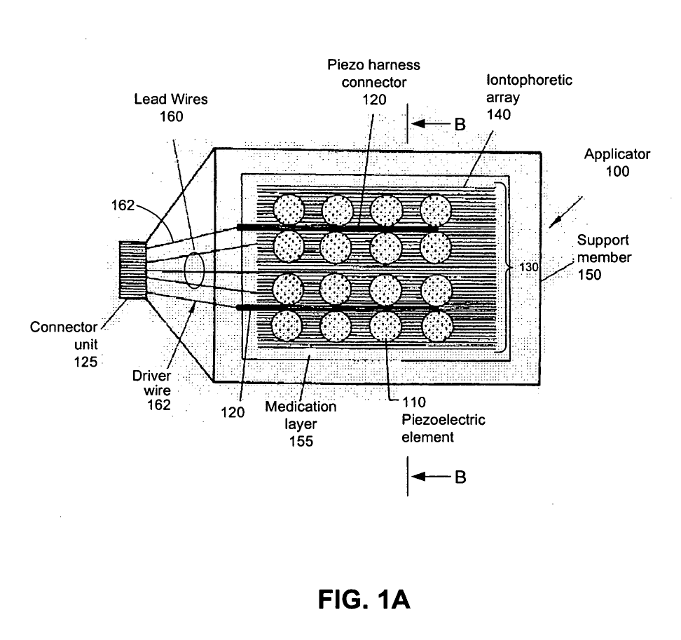

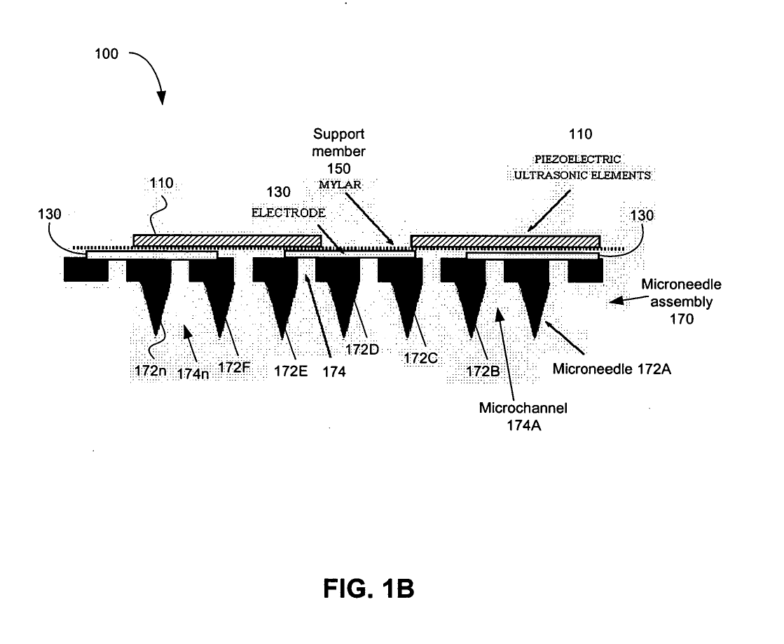

FIGS. 1A and 1B show a multichannel iontophoretic applicator 100 having a plurality of ultrasonic elements 110 and a microneedle assembly 170 that can be used to treat large dermal areas according to an embodiment of the present invention. As shown in FIG. 1A, multichannel iontophoretic applicator 100 includes a working electrode 140 having an array of iontophoretic electrodes 130. The working electrode 140 forms a closed circuit through the patient's body when current passes there through which promotes the penetration or absorption of an ionic medicament. The polarity of the working electrode 140 is selected based upon the polarity of the medicament to be administered. A grounding electrode (not shown) employed with the multichannel working electrode 140 may also cover an area of skin that is similar in size to the area covered by electrode 140.

Applicator 100 further includes a support member 150. In an embodiment, support member 150 comprises a flexible sheet or film forming a conductive matrix having a current distributing conductive layer, such as a metallic foil, a conductive rubber or resin film, carbon film or other conductive coating or electro-dispersive material. The conductive matrix is flexible so that it may be contoured to the body area on which it is placed and still cover a relatively wide area.

As shown in FIG. 1B, according to a feature of the present invention, applicator 100 includes a microneedle assembly 170 having a plurality of microneedles 172A-172n. A plurality of microchannels 174A-174n are interspersed between microneedles 172A-172n. Microneedles 172 can be distributed in a regular, irregular, custom or other type of pattern depending upon a particular application. Microchannels 174A-174n can also be distributed in a regular, irregular, custom or other type of pattern depending upon a particular application. In one example, microneedle assembly 170 is perforated and microchannels 174A-174n comprises perforations in microneedle assembly 170.

Microneedles 172 can be any type of microneedle including but not limited to a hollow and/or solid microneedles made of plastic, metal or other material. In one example, the needles are made of an inert nonconductor or semiconductor of sufficient hardness. Microneedles 172 can be any desired length and can have uniform or varying lengths depending upon a particular application. In one example, microneedles 172 have lengths between approximately 10-350 microns. These example materials and dimensions are illustrative and not intended to limit the present invention.

In an embodiment, microneedle assembly 170 is coupled to support member 150. In an example shown in FIG. 1B, electrodes 130 are attached to support member 150.

Microneedles 172 are attached directly or indirectly to electrodes 130 and support member 150. According to one feature, the groups of microneedles 172A-172n and microchannels 174A-174n are arranged or clustered with respect to corresponding electrodes 130. For example, the pitch or separation distance between adjacent microneedles 172 is less than the pitch or separation distance between adjacent electrodes 130.

In one embodiment, support member 150 further includes a medicament-carrying layer 155 attached to it, such as, by an adhesive. Medicament carrying layer 155 can be formed from a porous material about ¼ of an inch thick which can be a honeycombed sponge-like material with vertical cells to minimize cross flow or lateral dispersion of the medicament. An open-celled sponge-like material in the medicament-carrying layer 155 can be inert to the medicament or treatment agent being employed, as well as being noncorrosive and stable. Suitable materials include plastic pads, such as polyethylene, paper or cotton, porous ceramics, open-celled porous polytetrafluoro ethylene, other inert plastics, and open-celled silicone rubber, preferably with vertically aligned medicament-containing cells or tubes.

A separate medicament carrying layer 155 however is optional. In other embodiments, medicament can be part of or integral with support member 150. In still other embodiments, a medicament carrying layer can be part of, integral with, attached to, or externally applied to microneedle assembly 170 as described further below with respect to FIG. 1B.

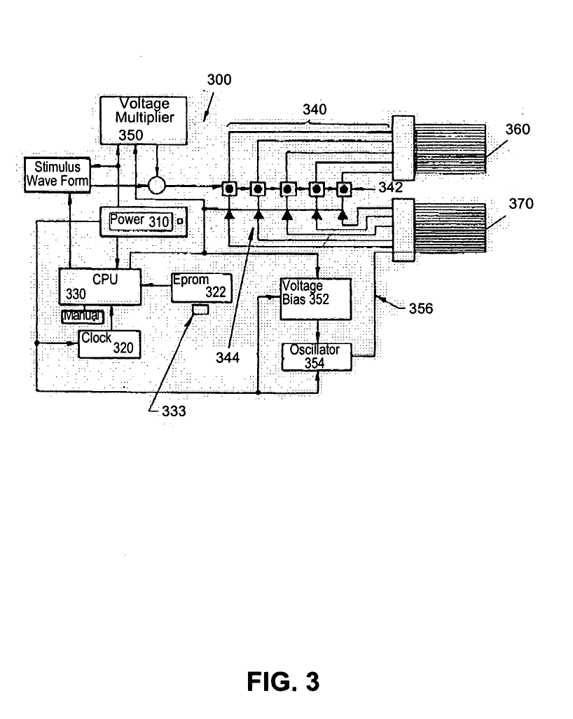

A ribbon connector (not shown in FIG. 1) connects an electrical power source (not shown in FIG. 1) to the multichannel electrode 140 and delivers the electrical current with multiconnectors in connector unit 125 to lead wires 160 that form the individual electrically conductive channels in the conductive array of iontophoretic electrodes 130. Each channel in the iontophoretic array 140 carries no more than 1 milliamp, but this is representative only and not intended to limit the present invention. The amount of current that flows to each channel is controlled by a control circuit (shown in FIG. 3) to prevent a tunneling effect from occurring. This prevents the flow of current along the path of least resistance through a lesion or skin rupture, for example, resulting in a burn to the patient at that location. The multichannel electrode 140 can employ a circuit pattern etched such as by laser or photoetching onto, for example, a metal coated Mylar® plastic sheet with each channel isolated to facilitate dispersion over a broad surface area.

Each channel formed by the lead wires 160 can be electrically driven simultaneously, in a sequential multiplex fashion, or in any other known or custom designed fashion. The use of simultaneous or parallel electrical current to each lead wire 160 in the array 140 would be employed, for example, in the application of medicament to burns where a wide area of dispersion is required. The iontosonic applicator 100 greatly improves the skin penetration by the medicament to actively deliver the medicament to either a wide regional area of the skin or to a specific lesion on the skin.

Ultrasonic elements 110 can be made of piezoelectric crystal elements or any other suitable piezoelectric material. Ultrasonic elements 110 may be mounted on a flexible electrode 140 by means of a suitable adhesive such as SILASTIC® brand of silicone adhesive. Driving oscillator connections 162 to the crystals can be photoetched onto a polymer sheet (e.g.; metalized MYLAR®) with perforations on the sheet that facilitate mounting of the ultrasonic elements 110. In this way, electrode 140 with ultrasonic and ionophoresis can be effective in moving insulin across skin, as well as antibiotics, antifungal, anti-inflammatory, blood pressure medication and cardiotropic drugs; either as direct drive, logic control timer drive or more elegantly as biofeedback control configuration. It is also effective in the treatment of wide field dermatological conditions, such as eczema, psoriasis and acne. It is also effective for ionic retention of skin hydrating media to facilitate skin hydration in cosmetic applications and in dermal exfoliation to drive medication into the skin in order to inflame the skin and cause the peeling of the external skin layer to stimulate reformation of collagen and collagen growth factors. The ionosonic applicator 100 can also prove useful for driving MINOXIDIL® or related compounds into the scalp to enhance hair growth and/or ameliorate baldness. The construction of ultrasonic elements 110 can be piezo-electric crystals, ceramics or distributed segments of piezo poly(vinylidenefluoride) film (KYNAR®). Further description of structure and operation of components in FIG. 1A can include the description provided in two earlier U.S. Pat. Nos. 5,658,247 and 5,538,503 to the same inventor, both of which are incorporated herein by reference in their entirety.

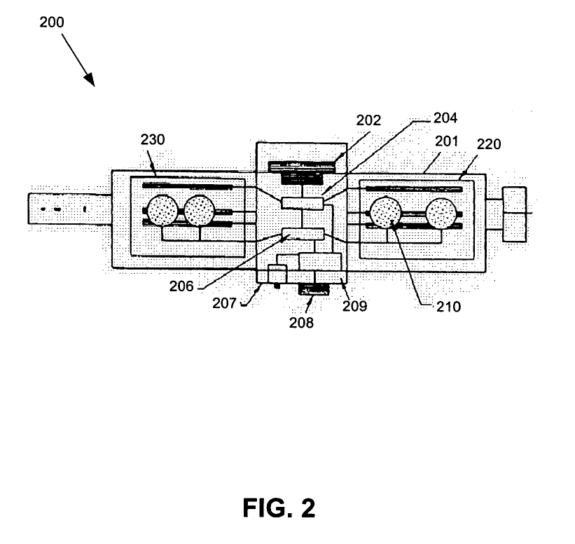

FIG. 2 shows multichannel iontophoretic-ultrasonic applicator 200 arranged in a band configuration for intradermal drug delivery. Applicator 200 can be adapted to include the microneedle assembly of FIG. 1B according to a further embodiment of the present invention. Applicator 200 incorporates applicator 100, which is coupled to a power source 202. The power source supplies energy for an iontophoretic driver 204 and an ultrasonic driver 206. A third driver, a manual driver 208 is also coupled to applicator 100. A CPU and clock 209 and an eprom and connector 207 are further included. Applicator 200 further includes piezo crystals 210, active electrode array 220 and neutral electrode array 230.

FIG. 3 shows a block circuit diagram of the iontophoretic medicator electrical control circuit 300 suitable for use with the multichannel ionosonic applicator 100 of FIG. 1 and the miniaturized ionosonic applicator 200 diagrammed in FIG. 2. The control circuit, generally indicated at 300, may be either integrated with the applicator electrode, as shown in FIG. 2, or boxed separately and including connection means adapted to electrically connect to the applicator to provide power to drive the applicator as shown in FIG. 1. The control circuit 300 is equipped with a power source 310 which may be either a battery or an isolated wall source. Further description of structure and operation of components in FIGS. 2 and 3 can include the description provided in two earlier U.S. Pat. Nos. 5,658,247 and 5,5,538,503 to the same inventor, both of which are incorporated herein by reference in their entirety.

The control circuit 300 is provided with a clock-operated timer switch 320 to preset the length of iontophoretic treatment mediated by an integral CPU 330. Once the length of time has been selected, a voltage multiplier 350 is utilized to provide the current to iontophoretically drive the medicament into the patient's skin. The current is set and administered until the end of the treatment period. When the clock 320 signals the end of the treatment period, the electrical current to electrode 370 is gradually terminated by a ramping down of the current to the patient to avoid abrupt change. The treatment program can be stored in the eraseable programmable read only memory device (EPROM) 322, or by an external program 333. Also in control circuit 300 are voltage bias 352, oscillator 354 and buffers 344.

Ribbon cable (not shown) provides a flexible connection to multichannel neutral electrodes 360 and active electrode 370 as indicated in FIG. 3, as well as delivering oscillator power for the piezoelectric crystals 110 mounted on the applicator electrode 140. Internal circuit board controls allow for frequency adjustment, adjustment of maximum current per iontophoretic channel (not to exceed 0.6 to 1.2 ma range), and internal control that will shut down any iontophoretic channel electrically performing outside a “normal” range of encountered biological impedance.

With further reference to FIG. 3, the block circuit diagram of the large area iontophoretic medicator control circuit is shown which can be employed with the multichannel ionosonic applicator of FIG. 2. An isolated current loop generator is employed to feed current to the individual channels in the multichannel electrode via the plurality of individual current loops 340 through current drivers 342. Each current loop drives one band or channel in the multichannel electrode. It has been found that 0.6 milliamps current flowing to each channel used within a wide field dispersion-grounding electrode, such as that shown in FIG. 1, provides a safe level for operating the ionosonic device. This level of current avoids the tunnelling effect of current flowing along the path of least resistance and concentrating in, for example, a lesion or skin rupture, resulting in a burn to the patient. This permits current to be distributed over the large area of the multichannel electrode to drive medicament through a patient's skin over a large dermal area. Depending upon the electrode configuration, this current level can vary from about 0.1 to about 1.2 milliamps. The novel introduction of distributed ultrasonic piezoelectric elements combined with the iontophoretic multi electrodes described above greatly enhances the rate of penetration of many molecules. The use of ionosonic applications to administer insulin transdermally now becomes feasible.

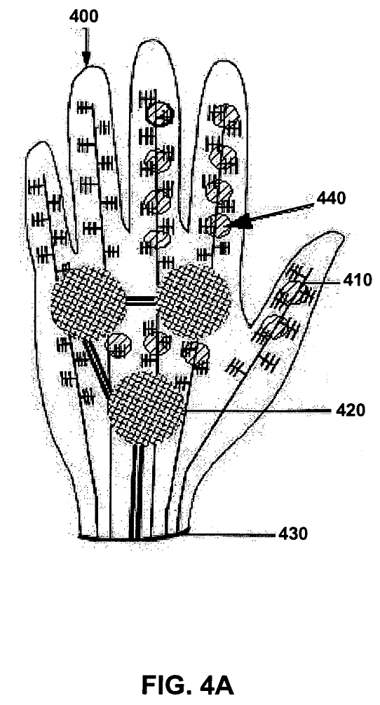

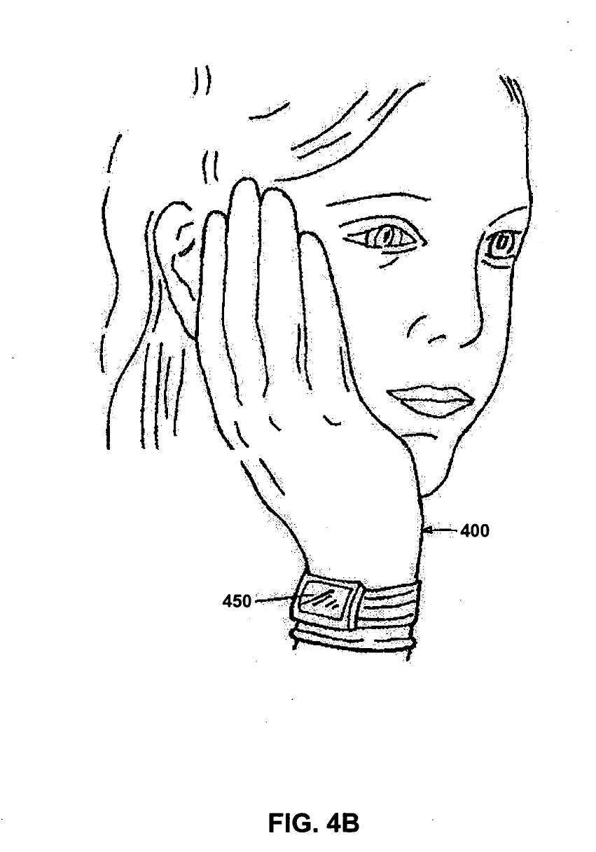

The multichannel ionosonic applicator 100 described above has many applications wherein it is desired to deliver a therapeutic agent only into the epidermis and dermis (i.e., intradermally). FIG. 4A is a palmar view of a glove 400 having a plurality of iontophoresis applicator electrodes 410 and return (neutral) electrodes disposed thereon and including a plurality of ultrasonic elements 420 disposed thereon. Microneedle elements (continuous or plurality of) are incorporated into the device as symbolically represented by 440. The micro channels within such elements are disposed in proximity of micro projections and are of sufficient thickness to serve as a medicament reservoir or suitable conductive medium where on one side is in electrical contact with the driving dispersion electrode and the other side is in direct contact with skin undergoing treatment. The electrodes 410 and piezoelectric elements 420 are in electrical connection with a power source (not shown in FIG. 4A) by a connector harness 430 affixed to the glove. FIG. 4B is a perspective view of a mammal, in this case, a human person, wearing an ionosonic glove applicator 400 of FIG. 4A and employing the device 400 to self-administer a cosmetic or therapeutic agent to the skin of the face thereafter the agent to be delivered to intradermal tissue. An operator assembly 450 is shown that includes, for example, the battery, or other power source, the electronics and ground electrodes.

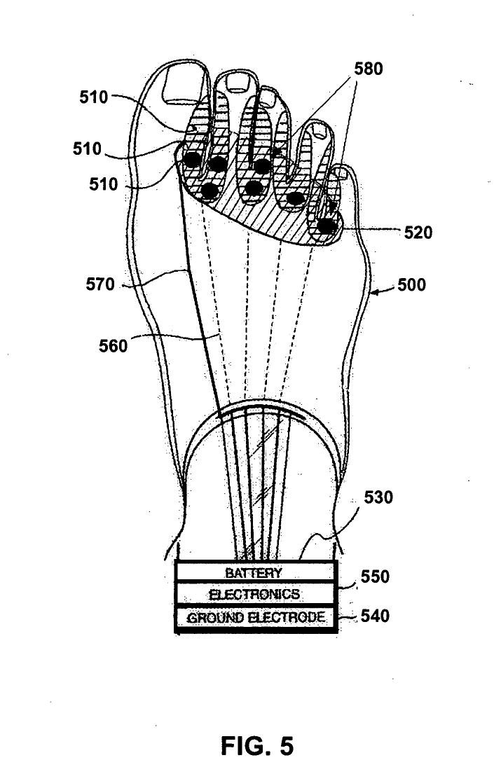

FIG. 5 shows an ionosonic bootie 500, in accordance with an embodiment of the present invention. Ionosonic bootie 500 includes a plurality of iontophoresis electrodes 510 and an ultrasonic electrode 520 disposed thereon for delivering an agent into the skin of the toes (FIG. 5). In one example, the agent can be an antifungal agent. The bootie 500 includes a power source 530, a grounded electrode 540 which is in electrical communication with the patient's skin distal from the toes, and an electronic circuit 550 which is preferably programmable and operable for providing a driving current to the iontophoresis electrodes 510 and piezo element 520 through conductors 560 and 570 respectively. A microneedle assembly having microneedles (continuous or plurality of) and microchannels are incorporated into the device 500 as symbolically represented by 580. The microchannels within such elements are disposed in proximity of microprojections and are of sufficient thickness to serve as a medicament reservoir where on one side is in electrical contact with the driving dispersion electrode and the other side is in direct contact with skin undergoing treatment.

In another embodiment, ionosonic bootie 500 can have a plurality of iontophoresis electrodes and at least one ultrasonic electrode disposed on an inner skin-contacting surface thereof that is operable for delivering an antifungal agent into one or more infected toenails. The bootie 500 includes a driver comprising a power source such as a battery and an electronic controller circuit, preferably programmable, operable for conducting a driving current to the iontophoresis electrodes and the ultrasonic piezo element(s) through electrical conductors, respectively. Microneedle elements (continuous or plurality of) are incorporated into the device. The microchannels within such elements are disposed in proximity of micro projections and are of sufficient thickness to serve as a medicament reservoir or suitable conducting medium where on one side is in electrical contact with the driving dispersion electrode and the other side is in direct contact with skin undergoing treatment. The antifungal agent may be applied directly to the infected nail prior to donning the boot or contained within a reservoir comprising the iontophoresis electrodes.

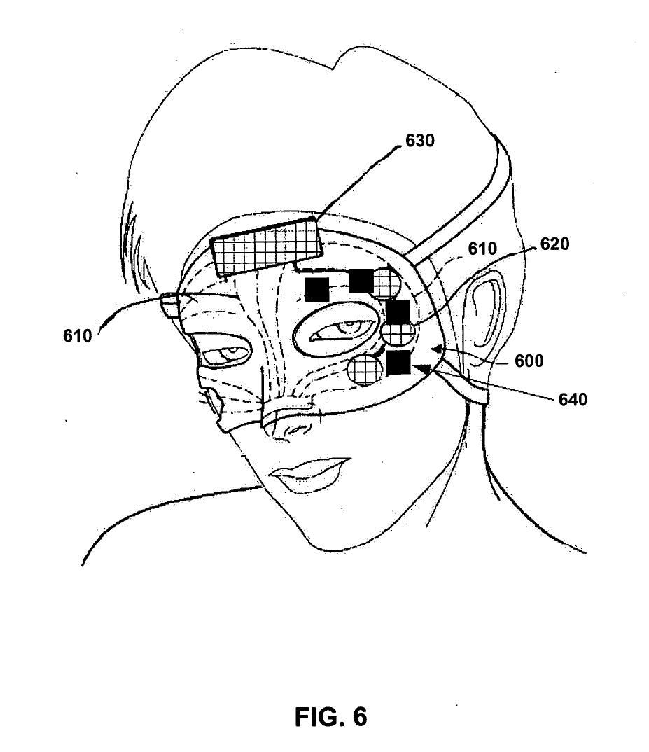

FIG. 6 is a schematic view of an ionosonic mask 600 similar in operation to the ionosonic bootie 500 and having a plurality of iontophoresis electrodes 610 and ultrasonic elements 620 disposed thereon for the intradermal delivery of a cosmetic or therapeutic agent to the skin of the face. The mask 600 includes a driver 630 operable for providing a driver current to the iontophoresis electrodes 610 and the piezoelectric ultrasonic elements 620. Microneedle assembly 170 including microneedles 172 and microchannels 174 (continuous or plurality of) are incorporated into the device as symbolically represented by 640. The micro channels 174 within such microneedle elements 172 are disposed in proximity of micro projections and are of sufficient thickness to serve as a medicament reservoir where on one side is in electrical contact with the driving dispersion electrode and the other side is in direct contact with skin undergoing treatment. In one example, the mask 600 is useful for the intradermal delivery of cosmetic/therapeutic agents for the treatment of wrinkles.

A particularly recalcitrant medical condition that is benign but unsightly is onychomycosis. Onychomycosis, also called ringworm of the nails, or tinea unguium, is a fungus infection of the nails causing thickening, roughness and splitting. The infection is caused by various fungal species including Trichophyton rubrum, Trichophyton tonsurans, Epidermophyton floccosum and Trichophyton mentagrophytes. Once these microorganisms establish themselves within or under a nail, eradication with current over-the-counter (OTC) antifungal agents is difficult, costly and time consuming and recurrences of the disease can be expected. Antifungal agents that are formulated for topical application, such as undecylenic acid, lotrimin and tolnaftate, are effective for treating fungal infections of the skin but, as presently formulated and administered, are ineffective for treating fungal infections of the nail.

Treatment regimens for onychomycosis include prolonged and sustained application of topical fungicidal creams and/or solutions directly to the infected nail(s) and/or systemic treatment with antifungal drugs such as griseoflulvin, terbinafine, and itraconazole. Some of the systemic treatments have undesirable side effects such as nausea, headache, photosensitivity, gastrointestinal intolerance, elevated liver enzymes, and undesirable drug interactions, making the value of the treatment questionable.

The embodiments of the multichannel ionosonic intradermal drug delivery device or applicator 100 discussed above are wearable and conform to the contour of the area of the infected or deformed skin, nail and/or other tissue to be treated.

The present invention provides novel delivery devices and methods utilizing iontonsonic and microneedle technologies. These delivery devices and methods can be used in a wide variety of embodiments and applications including, but not limited to, intradermal or transdermal delivery of one or more therapeutic agents toward or into a tissue (such as a skin or nail).

In an embodiment, an applicator can ionosonically drive an agent toward a targeted portion of a mammal's body. The applicator includes one or more piezoelectric elements, a plurality of iontophoresis electrodes, and a microneedle assembly. The microneedle assembly has a plurality of microneedles that can be placed at or near the targeted portion of a mammal's body. The one or more piezoelectric elements are coupled to the microneedle assembly. The plurality of microneedles move and vibrate the tissue in contact with the applicator in response to the changes in the piezoelectric element(s). Electrical current can be applied to the plurality of iontophoresis electrodes to further drive the agent toward the targeted portion of the mammal's body.

According to a feature, the microneedle assembly further includes a plurality of microchannels. The plurality of microchannels can be interspersed between the plurality of microneedles.

According to another embodiment, the iontophoresis electrodes are arranged at or near respective groups of microneedles and microchannels. In a multichannel embodiment, each iontophoresis electrode can be separate channel electrically isolated from other channels. A plurality of current drivers can be coupled to respective ones of the plurality of iontophoresis electrodes. In this way, each iontophoresis electrode comprises a separate electrical channel driven by a corresponding current driver.

According to a further embodiment, an applicator includes a support member arranged to support one or more piezoelectric elements relative to the microneedle assembly as well as the plurality of iontophoresis electrodes. In one example, a plurality of iontophoresis electrodes are imbedded within the support member.

In some embodiments, an agent can be included in a medicament. The medicament can be included in the applicator as part of a separate medicament layer, as part of the support member, as part of the microneedle assembly (for example in perforations in the microneedle assembly), or applied externally to exterior of the microneedle assembly. In each case, the agent is released or moved when the plurality of microneedles move and vibrate tissue in contact with the applicator in response to the at least one piezoelectric element, and when electrical current is applied to the plurality of iontophoresis electrodes to further drive the agent toward the targeted portion of the mammal's body.

In an embodiment, an ionosonic intradermal drug delivery device adapted to releasably attach to a mammal's body so that a skin-contacting surface of the device is adjacent to a targeted portion of the mammal's skin or nail. The device is operable for ionosonically driving a medicament across a skin-contacting surface of the device into a targeted portion of the mammal's skin or nail. The device includes (a) a medicament carrying layer in fluid communication with the skin-contacting surface of the device comprising a porous sheet impregnated with a medicament containing fluid, (b) an iontophoresis electrode in electrical communication with the medicament carrying layer providing means for iontophoretically driving the medicament into the targeted portion of the mammal's skin or nail(s) wherein the iontophoresis electrode comprises a plurality of electrode channels, each electrode channel of the plurality of electrode channels being electrically isolated from other electrode channels, the plurality of electrode channels having electrical connection means connected thereto adapted for simultaneous electrical connection of selected electrode channels to the same or different current sources, the each electrode channel being in electrical communication with the medicament carrying layer; (c) at least one piezoelectric element affixed to the iontophoresis electrode and overlying at least one of the iontophoresis electrode channels; and (d) at least one perforated non or partially conductive microneedle array layer interposed between the skin and iontophoretic dispersion electrode.

The medicament can be any type of medicament including, but not limited to: any medicament or combination of medicaments for treating or preventing microbial infection of skin or nail, treating or preventing acne, treating psoriasis, treating or preventing eczema or contact dermatitis or atopic dermatitis, treating or preventing onychomycosis, treating actinic keratoses, treating skin cancer, treating a wound, or treating an infection by a human papilloma virus. The medicament can be any type of medicament including, but not limited to, an anesthetic, therapeutic drug, or a non-therapeutic agent. The medicament can be in any form including, but not limited to, any type of cream or liquid.

In an embodiment, a method for the transdermal or intradermal self-administration of a medicament into a targeted portion of the skin or nail of an individual is provided. The method includes (a) presenting an ionosonic intradermal drug delivery device; (b) impregnating a porous sheet with a medicament and placing the porous sheet in contact with the targeted portion of the individual's skin or nails; and (c) causing an electrical current to flow through the electrode channels of the iontophoresis electrode to drive the medicament into the targeted portion of the skin or nail.

The drug delivery device of the present invention incorporates a multichannel iontophoresis electrode array on a single application electrode and includes one or more piezoelectric elements capable of generating vibratory ultrasonic fields to further enhance the penetration of therapeutic agents into the skin. Attached to this device and in direct contact with the skin is a plurality of multineedle array elements constructed from a non-conductor or semiconductor such as silicone or silicone dioxide. The protruding elements may be hollowed or geometric as either of those configurations will work. In several of our applications the simpler to manufacture non-hollowed version may be preferred. The base of such protrusion array may be rigid or flexible.

Although concurrent use of ultrasound has been described to enhance transdermal penetration of a microneedle device there has not been a description of concurrent ultrasound and ionophoresis especially a multichannel dispersive one where each channel of the ionophoretic driver is controlled by its own current limiter circuit. It is well understood that microneedles between 5-250 microns in length but predominantly in the 100-200 micron length penetrate the corneum stratum that is the main layer that impedes the molecular penetration of the skin. Whereas the standard microneedle devices described elsewhere have been plagued by nonuniform penetration of the stratum corneum, and ionophoretic drivers have been plagued by tunneling effects leading to burns and unpredictable therapeutic effects, embodiments of the present invention can overcome these problems and have improved clinical performance. As recognized first by the inventor, at the site of application combining microneedle elements with ultrasonic elements improves the uniformity and efficacy of the penetration and breakdown of the obstructing stratum corneum making each one of those respective technologies mutually synergistic in decreasing the stratum corneum transport obstructive quality. The ultrasound not only helps disperse the medication within the electrode matrix but also improves the imbedding and penetration of the sharp protruding elements. The protruding elements are also believed to further create microcavitation effects that further disrupt the stratum corneum barrier behavior. Once the barrier is partially compromised by microneedles with ultrasonic overlay the penetration of the dermis is further accelerated by the multichannel ionophoretic dispersion electrode that is integrated together with the ultrasonic and microneedle elements. The ultrasonic elements work synergistically with ionophoresis as they decrease polarizing effects and disperse localized ph issues and facilitate neutralization by imbedded buffering molecules. Combining such three somewhat distinct technology elements (ionophoresis, ultrasound, and microneedles) into a single application device greatly enhances the functionality and performance of such transdermal devices that use a single technology or a combination of only two of such elements. In an embodiment the device is programmable and includes a CPU and programming means such as an EPROM. The CPU and EPROM enable the operating parameters of each of the electrodes comprising the multichannel electrode to be independently controlled and changed.

Multichannel iontophoretic and ionosonic drug delivery devices disclosed herein have numerous clinical applications and such devices are further enhanced by integrating microneedle arrays into such devices. A simple application of such a device is the treatment of a (very painful) herpetic infection affecting a large skin area and along a sensory nerve regional distribution referred to as Herpes Zoster or, more commonly, “Shingles.” Each channel of the multichannel device has a separate current limited driver circuit to prevent a runaway tunneling effect and make possible the application of therapeutic agent to a larger skin area yet retain control of an even distribution and penetration.

A related disease is viral warts and skin papillomas. Current modality involves surgery, laser burn, chemical burn, electrical burn and freezing. Embodiments of the present invention can be used in new therapeutic approaches to skin disorders and systemic applications, whether they are a nuisance category, one of vanity, health or serious life-threatening nature.

In an embodiment, a drug delivery device of the present invention incorporates a multichannel iontophoresis electrode array on a single application electrode and includes one or more piezoelectric elements capable of generating vibratory ultrasonic fields to further enhance the penetration of therapeutic agents into the skin. Attached to this device and in direct contact with the skin is a plurality of multineedle array elements constructed from a non-conductor or semiconductor such as silicone or silicone dioxide. The protruding elements may be hollowed or geometric as either of those configurations will work. In several applications a simpler to manufacture solid or non-hollowed version may be used. The base of such protrusion array may be rigid or flexible.

According to a further feature, concurrent use of ultrasound and ionophoresis is provided in an applicator and methods therefor. In a further feature, a multichannel dispersive applicator where each channel of a ionophoretic driver is controlled by its own current limiter circuit is provided. It is well understood that microneedles between 5-250 microns in length but predominantly in the 100-200 micron length penetrate the corneum stratum that is the main layer that impedes the molecular penetration of the skin. Whereas microneedle devices described elsewhere have been plagued by nonuniform penetration of the stratum corneum and ionophoretic drivers have been plagued by tunneling effects leading to burns and unpredictable therapeutic effects, an advantage of the embodiments of the present innovation are that they can improve significantly the clinical performance of such devices making many such devices now clinically possible. The inventor contends that at site of the application by combining microneedle elements with ultrasonic elements according to embodiments of the present invention can improve the uniformity and efficacy of the penetration and breakdown of the obstructing stratum corneum making each one of those respective technologies mutually synergistic in decreasing the stratum corneum transport obstructive quality. The ultrasound not only helps disperse the medication within an electrode matrix but also improves the imbedding and penetration of the sharp protruding elements. The protruding elements are also believed by the inventor to further create microcavitation effects that further disrupt the stratum corneum barrier behavior. Once the barrier is partially compromised by microneedles with ultrasonic overlay the penetration of the dermis is further accelerated by the multichannel ionophoretic dispersion electrode that is integrated together with the ultrasonic and microneedle elements. The ultrasonic elements work synergistically with ionophoresis as they decrease polarizing effects and disperse localized ph issues and facilitate neutralization by imbedded buffering molecules. It is the inventor's observation and contention that combining such three somewhat distinct technology elements (ionophoresis, ultrasound, and microneedles) into a single application device greatly enhances the functionality and performance of such transdermal devices that use a single technology or a combination of only two of such elements. In a further embodiment the device is programmable and includes a CPU and programming means such as an EPROM. The CPU and EPROM enable the operating parameters of each of the electrodes comprising the multichannel electrode to be independently controlled and changed.

It is provided a intradermal or transdermal drug delivery device with greatly improved penetration parameters and, more particularly, to a programmable multichannel iontophoresis electrode, combined with ultrasonic and microneedle arrays that greatly and synergistically operate for delivering a therapeutic agent to and through the skin. The enhanced transdermal delivery lends itself to the design of timed and programmable devices that can be combined with physiological sensors that can deliver the therapeutic through the skin in greater efficacy and with lesser toxic side effects.

Novel multichannel ionosonic devices with microneedle arrays incorporated into the devices for transdermal and intradermal delivery as well as transdermal serum sampling and diagnostic assay are described. In a preferred embodiment, the ionosonic device includes a multichannel ionophoretic driver used in combination with a multichannel dispersion electrode integrated with ultrasonic elements and microneedle array elements mounted on a single application electrode. The ionosonic-microneedle transdermal device can be configured in a variety of shapes and structural flexibility for the treatment of skin and systemic disorders through the intradermal and transdermal delivery of one or more of a variety of therapeutic and modulating agents. Because of enhanced transdermal penetration this device offers the transdermal delivery of therapeutic peptides is getting closer to reality. The devices described herein deliver the therapeutic agents to the targeted diseased area as well as systemic levels obviating the need for oral ingestion, the associated side effects and as in the case of peptides bypasses the hydrolyzing digestive enzymes that make such agents ineffective when taken by mouth.

### Additional Applications and Discussion

A discussion of skin disorders is appropriate at this time. There are a number of clinical disorders that afflict the dermal/epidermal layer that can be classified into inflammatory disorders exemplified by bacterial infection (e.g., Staphylococcus infection), viral infection (e.g., herpetic infestation), immuno reactive inflammatory disorders such as allergic welts, fungal infestation of skin and nails (e.g., monilia and onychomycosis (fungal infections of the nails and nail beds)) that chronically infects nails on both hands and feet. There also exist a variety of idiopathic inflammatory disorders such as localized vasculitis and number of rare blistering reactions, localized alopecia (loss of hair growth) and hypo pigmentation (vitiligo). Even for idiopathic disorders there are topical agents that may have some benefit but they are currently severely restricted by lack of penetration. The multichannel and ionosonic technology described herein when combined with application electrode and existing or emerging therapeutic agent can vastly improve the efficacy of such treatments yet avoid systemic toxicity when a therapeutic agent is ingested in order to act regionally. Incorporation of microneedle elements further enhances penetration to a degree that it opens therapeutic modalities for the transdermal delivery of peptides. Such new treatment approaches would open therapeutic options for existing compounds whose ingestion toxicities have not been tested, as they will be used topically and regionally with minimal systemic absorption.

A multichannel ionophoretic driver for transdermal delivery of therapeutic agents and applications thereof, have been previously patented by the present inventor. For example, a smokeless cigarette to help individuals cease smoking has been disclosed in U.S. Pat. No. 5,331,979 by the present inventor. The smokeless cigarette is based on the ionophoretic transport of nicotine through the mouthpiece of the device; the device having the general size and appearance of a cigarette. Upon contact with the lips, the facilitated transport of nicotine is activated and inhalation of smoke or other carcinogens is avoided. A wearable iontophoretic drug delivery device for substance abuse detoxification is disclosed in U.S. Pat. No. 5,538,503 to the present inventor.

A decade ago, animal studies demonstrated that ultrasound has a beneficial effect in improving penetration of skin in an animal model. The mechanism is thought to involve the disarray of the epidermal layer and perhaps dermal/epidermal microcavitation effects. As mentioned above, U.S. Pat. No. 5,538,503 by the present inventor discloses an apparatus for the programmable iontophoretic or iontophoretic-ultrasonic (ionosonic) transdermal delivery of medication across the skin or other biological membrane. In one embodiment, the programmed delivery of medicament is accompanied by an electrical stimulus for treating substance abuse. The apparatus can be adapted for large dermal area application or for a smaller area of application, depending on the choice of specific applicator electrode employed; In a preferred embodiment, the apparatus, disclosed in U.S. Pat. No. 5,658,247, comprises a multichannel iontophoretic applicator electrode. Multiple piezoelectric elements are mounted on the iontophoretic electrode. The combination of ultrasonic vibration and iontophoresis improves the penetration of medicament through the skin or mucous membrane underlying the electrode that can be programmed and controlled by a CPU through the use of an EPROM. A wearable embodiment of an ionosonic or iontophoretic drug delivery system employing programmability may be used to produce response conditioning useful for the outpatient treatment of obesity, nicotine detoxification, and narcotic addiction detoxification.

Such a multichannel iontophoretic electrode with suitable multichannel driver device combined with micro needles or dermal pretreatment protocol will in itself improve transdermal transport. Concurrent incorporation of ultrasonic elements will further enhance the performance of such a device. The performance of a multichannel iontophoretic driver will be enhanced by compromising the integrity of the keratin barrier of the skin (by use of chemical or abrasive agents) as well as micro needles. The concurrent use of ultrasound integrated into the same application electrode further enhances performance of such devices. Combining such electrodes with ultrasound to create a microneedle-ionosonic device further enhances transport parameters over and above the performance levels achieved by a single technology, or a double technology. By combining the multichannel iontophoretic device (electrodes and electronics) with micro needles and with or without ultrasound the inventor achieves further performance enhancement over previously described multichannel iontophoretic technology and over previously described ionosonic technology.

Although transdermal delivery systems that are efficacious, cost effective, reliable and not painful are one application of the technology discussed herein there are never the less skin diseases and infections that may be treated by the intradermal application of a therapeutic or modulating agent. In addition to treating skin infections, there are proliferative disorders of the skin including early neoplastic and preneoplastic changes such as actinic keratosis or even frank basal cell carcinoma that may be treated by the intradermal delivery of a therapeutic agent. Widespread multiple dysplastic neoplastic lesions are now treated with 8-12 week application of topical antimetabolites such as 5FU. There are neoplastic and dysplastic disorders. There are also a number of hypoplastic and atrophic changes in the skin (many consider psoriasis a proliferative disorder) that may be treated by intradermal drug delivery. The current psoriasis treatments can be greatly enhanced and even help the patient self-treat if a device enabling increased local penetration of a therapeutic agent such as those medications that currently applied topically applied but limited in their efficacy by their lack of penetration into the psoriasis lesions.

Although wound healing is a natural process, a disturbance of the normal process forms a pathological condition that is of great clinical relevance. A clear-cut example is the post-burn wound contracture that requires extensive surgical intervention and grafting. The constant contracture of a scar creates distortion and interferes with normal function. Certain ethnic groups are also subject to keloids, which is another example of the healing process that continues to deposit new collagen when it is no longer needed for the healing process. The opposite polarity of this hyperactive wound healing is a deficient wound healing process. Wound dehiscence or insufficient collagen deposition where the wounds do not heal appropriately is an example of such a process often afflicting elderly, malnourished, immunocompromised patients and, for some, an idiopathic event. Other medical conditions may contribute to some of the aforesaid wound-healing disorders and there are a variety of agents that are designed to modulate this problem. Embodiments herein may strive to deliver these agents topically or systemically, alternatively, embodiments may also provide a device that can deliver high doses of therapeutic agents to the dermal and the dermal junction which is the therapeutic area that needs to receive the agent in order to respond.

Yet a further category of skin disorders that may benefit from the provision of an intradermal drug delivery device is the anti-aging application. Although the aging process on the skin is a normal progression, society does not accept this process and there are a variety of products designed to ameliorate the aging process. There is a current need for an intradermal drug delivery device that potentiates existing agents significantly through the delivery into the dermis at high levels. Substances that may be efficacious in reducing the effects of aging are collagen deposition stimulants, vitamin C, anti-oxidants, chemotactic peptides and growth factors that may not only modulate new collagen formation but also wound healing.

Wound healing is a complex process modulated by many enzymes, peptides, and cellular activation. The sequence of these biochemical and cellular events is timed with the precision of an orchestra. Nevertheless, burns and other conditions create numerous challenges where intradermal drug delivery devices may prove beneficial. During the early stages of burn management the eschar has to be surgically removed (an exceptionally painful process), and wound dressings must be constantly maintained to prevent fluid loss and prevent infection. This requires extended hospital stay, multiple dressing changes, surgical debriedment and than surgical grafting. It is, therefore, desirable to provide an intradermal drug delivery device that can be applied to saturate the eschar with antibiotic/antifungal compounds so that the eschar can be used as a burn dressing until enzymatic separation occurs. A device that combines a multichannel dispersive ionophoretic driver, ultrasonic elements and microneedle arrays into a single conforming application device will have unique therapeutic advantages not only in transdermal and reverse serum sampling applications but also in numerous intradermal applications described herein. By the same token the enhanced capability to deliver peptides to and through the skin opens numerous novel clinical therapeutic modalities.

These secondary-healing stages could further be modulated by providing an intradermal drug delivery device operable for driving enzymes, interleukins, growth factor peptides and monoclonal antibodies targeted at specific healing stage enzymatic suppression into the skin at the burn site. Such technology may one day save significant costs and pain of burn management as many patients could be shifted from inpatient management to outpatient. A more challenging phase of burn wound management happens after successful grafting and closure of wounds. The scar tissue continues contract and creates a painful and disfiguring remainder of the primary burn. These patients are subjected to many years of scar excision and grafting in the attempt to return them to normal life with satisfactory limb function and acceptable appearance. The present inventor has conducted research in an animal model demonstrating a decrease of post injury contracture by use of systemic lathyrogenic agents. It would be desirable to provide an intradermal drug delivery device operable for driving a lathyrogenic agent directly into the contracting to achieve a significant benefit on this horrible sequela of burns without incurring the systemic toxicity when such agents are taken systemically. Since their action is on weakening the collagen, when taken systemically such agents can have life threatening side effects and therefore local wound therapeutic levels are seldom achieved.

A recent treatise on wound healing alludes to pharmacological modulators of wound healing including: (a) anti-inflammatory drugs such as aspirin, ibuprofen, naproxen and antihistamines e.g., diphenhydramine; (b) protein synthesis inhibitors including topical or intra-lesional steroids, colchicines, 5-flurouracil; (c) lathyrogenic agents such as BAPN, pencillamine or putrescine; (d) proteolytic enzyme synthesis stimulators include interleukin-1, calmodulin or protein kinase C inhibitors; and (e) calcium channel blockers such as verapamil or nifedipine. Accordingly, it is desirable to provide an intradermal drug delivery device to use for the benefit of burn treatment and wound healing modulation as well as the prevention of long-term contracture.

There is currently a massive research effort to identify a new class of peptide therapeutic agents. Peptide agents such as botox, epidermoid derived growth factors, chemo tactic agents, interleukins, etc have to be delivered by injection. Unfortunately such new products are peptides, and when taken systemically are not absorbed because they are broken down by stomach acid. Topically they have minimal penetration and are, therefore, not readily efficacious. It is desirable to provide an intradermal drug delivery device operable for driving small peptides directly into the diseased area in the skin. An emerging technology of regional delivery of gene activators or suppressors to modulate a disease process would potentially benefit from delivery technology described herein.

The identification of particular therapeutic agents for the treatment of skin disorders begins by choosing molecular agents from pharmacological compounds, herbal compounds, or chemical compounds that demonstrate reasonable clinical efficacy. There are currently a number of available anti-inflammatory agents, such as steroids, antibiotic agents, anti-viral agents, anti-metabolite agents, immune modulating agents and anti-aging agents that modulate collagen metabolism in the dermis. Some of those compounds are patented and many are not. There is a need for an intradermal drug delivery device operable for delivering such agents to the desired diseased location (i.e., a targeted area of skin) without discomfort and in sufficient concentration to greatly enhance their therapeutic efficacy.

The practical limitations of existing topical ointments and formulations that contain therapeutic agents has been the minimal penetration of the epidermal layer and the fact that many topical agents do not sufficiently penetrate the dermis. Some existing therapeutic agents require 6-12 weeks of repeated application and general patient compliance is low. The topical applications are also limited by the fact that ointments, as soon as they evaporate, the penetration by passive diffusion stops. Researchers have modulated the depth of penetration through occlusion and the incorporation of various carrier molecules that facilitate transdermal and intradermal transfer of the therapeutic agent. There is an ongoing need for an intradermal drug delivery device that can drive therapeutic agents into the dermis. The device described herein greatly improves the penetration of therapeutic agents through and into the dermis.

### CONCLUSION

While various embodiments of the present invention have been described above, it should be understood that they have been presented by way of example only, and not limitation. It will be apparent to mammals skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

What is claimed is:

1. An applicator that can be used to ionosonically drive an agent toward a targeted portion of a mammal's body, the applicator comprising:
at least one piezoelectric element;
a plurality of iontophoresis electrodes; and
a microneedle assembly having a plurality of microneedles that can be placed at or near the targeted portion of a mammal's body, and in direct contact with skin or tissue;
wherein the at least one piezoelectric element is coupled to the microneedle assembly such that the plurality of microneedles can move and vibrate tissue in contact with the applicator in response to the at least one piezoelectric element, and
wherein electrical current can be applied to the plurality of iontophoresis electrodes to further drive the agent toward the targeted portion of the mammal's body.

2. The applicator of claim 1, wherein the microneedle assembly further includes a plurality of microchannels.

3. The applicator of claim 2, wherein the plurality of microchannels are interspersed between the plurality of microneedles.

4. The applicator of claim 1, wherein each iontophoresis electrode is arranged with respect to respective groups of the microneedles and the microchannels.

5. The applicator of claim 1, further comprising: a support member arranged to support the at least one piezoelectric element relative to the microneedle assembly.

6. The applicator of claim 5, wherein the support member further supports the plurality of iontophoresis electrodes.

7. The applicator of claim 6, wherein the plurality of iontophoresis electrodes are imbedded within the support member.

8. The applicator of claim 5, wherein the support member further includes a medicament carrying portion that can release the agent when the plurality of microneedles move and vibrate tissue in contact with the applicator in response to the at least one piezoelectric element, and when electrical current is applied to the plurality of iontophoresis electrodes to further drive the agent toward the targeted portion of the mammal's body.

9. The applicator of claim 1, further comprising a medicament carrying member coupled to the microneedle assembly.

10. The applicator of claim 1, wherein the microneedle assembly is perforated and includes a medicament carrying portion that can release the agent when the plurality of microneedles move and vibrate tissue in contact with the applicator in response to the at least one piezoelectric element, and when electrical current is applied to the plurality of iontophoresis electrodes to further drive the agent toward the targeted portion of the mammal's body.

11. The applicator of claim 1, wherein medicament having the agent can be applied to the exterior of the microneedle assembly prior to placing the applicator in contact with tissue.

12. The applicator of claim 1, wherein each iontophoresis electrode comprises a respective electrical channel.

13. The applicator of claim 1, further comprising a plurality of current drivers coupled to respective ones of the plurality of iontophoresis electrodes, wherein each iontophoresis electrode comprises a respective electrical channel driven by a separate respective current driver.

14. An ionosonic intradermal drug delivery device adapted to releasably attach to a mammal's body so that a skin-contacting surface of said device is adjacent to a targeted portion of the mammal's skin or nail, said device being operable for ionosonically driving a medicament across said skin-contacting surface of said device into said targeted portion of the mammal's skin or nail, said device comprising:
(a) a medicament carrying layer in fluid communication with said skin-contacting surface of said device comprising a porous sheet impregnated with a medicament containing fluid;
(b) an iontophoresis electrode in electrical communication with said medicament carrying layer providing means for iontophoretically driving said medicament into the targeted portion of the mammal's skin or nail(s) wherein said iontophoresis electrode comprises a plurality of electrode channels, each electrode channel of said plurality of electrode channels being electrically isolated from other electrode channels, said plurality of electrode channels having electrical connection means connected thereto adapted for simultaneous electrical connection of selected electrode channels to the same or different current sources, said each electrode channel being in electrical communication with said medicament carrying layer;
(c) at least one piezoelectric element affixed to said iontophoresis electrode and overlying at least one of said iontophoresis electrode channels; and
(d) at least one perforated non or partially conductive microneedle array layer interposed between the skin and iontophoretic dispersion electrode.

15. The ionosonic intradermal drug delivery device according to claim 14 wherein said at least one medicament is operable for treating or preventing microbial infection of skin or nail.

16. The ionosonic intradermal drug delivery device according to claim 14 wherein said at least one medicament is operable for treating a wound.

17. The ionosonic intradermal drug delivery device according to claim 14 wherein said at least one medicament is operable for treating or preventing acne.

18. The ionosonic intradermal drug delivery device according to claim 14 wherein said at least one medicament is operable for treating psoriasis.

19. The ionosonic intradermal drug delivery device according to claim 14 wherein said at least one medicament is operable for treating or preventing eczema or contact dermatitis or atopic dermatitis.

20. The ionosonic intradermal drug delivery device according to claim 14 wherein said at least one medicament is operable for treating or preventing onychomycosis.

21. The ionosonic intradermal drug delivery device according to claim 14 wherein said at least one medicament is operable for treating actinic keratoses.

22. The ionosonic intradermal drug delivery device according to claim 14 wherein delivery of the at least one medicament is operable for treating skin cancer.

23. The ionosonic intradermal drug delivery device according to claim 14 wherein said at least one medicament is an anesthetic.

24. The ionosonic intradermal drug delivery device according to claim 14 wherein said at least one medicament is operable for treating an infection by a human papilloma virus.

25. A method for the intradermal self-administration of a medicament into a targeted portion of the skin or nail of an individual comprising the steps of:
(a) presenting an ionosonic intradermal drug delivery device in accordance with claim 1;
(b) impregnating said porous sheet with a medicament and placing said porous sheet in contact with said targeted portion of the individual's skin or nails; and
(c) causing an electrical current to flow through said electrode channels of said iontophoresis electrode to drive the medicament into the targeted portion of the skin or nail.

26. A device where the medicament containing layer is structurally integrated with the application electrode whereby the medication is impregnated within the lumen of the constructed microchannels

27. The ionosonic intradermal drug delivery device according to claim 14, wherein said at least one medicament is an agent for gene therapy.
